(19) United States
(12) Patent Application Publication
(10) Pub. No.: US 2008/0077429 A1
(43) Pub. Date: Mar. 27, 2008

(54) Portable instructional device

(76) Inventors: Matthew T. Coe, Annandale, NJ (US); Richard Costa, Bedminster, NJ (US); Fred Pether, St. Petersburg, FL (US)

Correspondence Address: Allen R. Kipnes, Esq.;WATOV & KIPNES, P.C., P.O. Box 247, Princeton Junction, NJ, 08550, US

(21) Appl. No.: 11/525,259

(22) Filed: Sep. 22, 2006

(51) Int. Cl. G06Q 10/00; A61B 5/00

(52) U.S. Cl. 705/2; 600/300

(57) ABSTRACT

There is provided a portable instructional device for communicating medically relevant information to a patient on-premises during the course of treating, diagnosing or preventing disease, symptoms, or conditions thereof. The instructional device includes a display portion with a touch sensitive surface having at least one activation area operable by the patient, a memory for storing at least one portion of the medically relevant information wherein each of the at least one portion of the medically relevant information is associated with a particular activation area, an audio assembly connected to the memory for producing sound, wherein the audio assembly is adapted for receiving a selected portion of the medically relevant information from the memory corresponding to the activation area triggered by the patient to convert the medically relevant information into audio form, and an electronic article surveillance tag secured to a portion of the device for detecting the removal of the device to an off-premise area.

### FIELD OF THE INVENTION

The present invention is relates generally to portable instructional devices for educating people, and more particularly a portable instructional device for educating or instructing patients about medically relevant information such as prescribed medical treatments, pharmaceutical products including medications, and medical devices on-premises such as in a physician or pharmaceutical setting. Such an instructional device is intended to convey through visual and audio means medically relevant information to assist the medical profession including physicians, pharmacists, and other healthcare professionals in assisting patients in the diagnosis, treatment and prevention of medical conditions.

### BACKGROUND OF THE INVENTION

During the course of medical treatment, the physician is expected to provide the patient with sound medical advice and information, and ensure the patient's information needs are met. Such medical advice may include information about a medical treatment, procedure, medication, medical devices and other medical products. This information allows the patient to make an informed decision, and enhances patient compliance and awareness. Similarly in a pharmacy setting where medical prescriptions are filled, the pharmacist is obligated to provide the necessary medical information concerning prescription medications and devices dispensed to the patient.

Unfortunately today, medical resources have become increasingly limited as the population ages. Physicians are experiencing greater pressure to take in more patients, thereby reducing the time the physician can spend with each patient. This greatly diminishes the information providing capacity of the physician. Other medical personnel and healthcare professionals including pharmacists are encountering the same burdens. Patient access to medical information has become more difficult to obtain. Patients may not appreciate the importance of complying with medical prescriptions particularly when they are not provided the opportunity to be educated about their treatment regimen, product efficacy, or even adverse drug side effects.

An estimated 70% of all medical prescriptions go unused, resulting in $77 billion in excess healthcare costs each year. It is estimated that 40% of patients who receive outpatient drug therapy will encounter a treatment failure or a new medical problem as a result of noncompliance with the prescribed treatment regimen. Noncompliance has been implicated in 125,000 deaths and up to 20% of all hospital and nursing home admissions a year. Patient anxiety about medications and medical treatments has also been found to contribute to the patient noncompliance. Increasing accessibility of information to patient that is easy to understand, may alleviate much of the anxiety, and improve compliance.

Accordingly, there is a need for a portable instructional device, which is designed to effectively communicate vital medical information in association with a pharmaceutical product, medication, medical treatment or medical device administered to the patient on-premises. Such an instructional device would be distributed in combination with the associated pharmaceutical product, medication, medical treatment or medical device in a physician or pharmacy setting. It would be a still further advantage in the art if the portable instructional device is designed with a compact and lightweight profile for convenient carry and storage, and is capable of providing easy access to the information contained therein, preferably in a discreet manner, while at the same time promoting patient compliance and safety.

### SUMMARY OF THE INVENTION

The present invention relates generally to a portable instructional device designed to convey important information to a user on-premises through interaction with a touch-activated visual/sound interface. The portable instructional device of the present invention provides the user with a simple information conveying tool suitable for educating and instructing patients about the corresponding medical treatment or medication they are receiving, all within a device that is compact and portable so that is may be readily carried by the patient. The instructional device of the present invention may be conveniently dispensed or distributed to a patient waiting on-premises, for example, in a physician- or a pharmacy setting. The instructional device of the present invention is intended for distribution prior to receiving the prescribed medical product or treatment, in order to answer most, if not, all of the questions the patient may have. The instructional device of the present invention is useful for enhancing compliance and facilitating proper usage, dosing, and treatment.

The instructional device of the present invention is particularly suited for use by patients on-premises at a physician's office or at a pharmacy. The patient may use the instructional device while waiting for a prescription order, waiting for the results of a medical test, waiting for post-exam consultation, and the like. The instructional device of the present invention can be used to assist the physician or pharmacist by providing the patient with the most up to date information relevant to the medical treatment, medication, medical device and the like, and ease the patient care load and time constraints on the physician or pharmacist. The instructional device of the present invention is not intended to be a substitute for the physician or pharmacist, but to be used as a tool by the physician or pharmacist to provide information.

The instructional device further includes an electronic tag to prevent the device from being removed to an off-premise area. The electronic tag or embedded sensor with special electronic or magnetic characteristics is furnished with the instructional device. Commercially available detectors capable of sensing such electronic tags may be located at the exit points of the premises to prevent the patient from inadvertently removing the instructional device to an off-premise area. Such electronic tags are known in the art and are typically used in tag and alarm systems also known as electronic article surveillance systems.

Optionally, the instructional device may include a wireless or radio paging mechanism that allows the patient using the device to be paged or notified that a physician or pharmacist is available to assist. In this manner, the patient may use the instructional device while waiting to see the physician or pharmacist anywhere on the premises. Such radio paging mechanisms are known in the art and are used in radio paging systems comprising a centralized control station with a page transmitter, and for example, a plurality of buttons which may be pressed individually to send a page to one of an associated plurality of corresponding paging mechanism.

The portable instructional device of the present invention comprises an audio assembly and patient operable or interactive display area operatively associated with the audio assembly to facilitate access to medical information useful to the patient including instructions or details such as, for example, dosage regimen and adverse side effects. The portable instructional device of the present invention is especially suitable for use by patients who may experience difficulty reading print material or have queries about their medical treatment or medication. The instructional device may be conveniently dispensed or distributed in a physician- or a pharmacy-patient setting. The patient may operate the device while waiting for the prescribed medication at the pharmacy, or during a medical appointment in the physician's office.

In one aspect of the present invention, there is provided a portable instructional device for communicating medically relevant information to a patient on-premises during the course of treating, diagnosing or preventing disease, symptoms, or conditions thereof, the instructional device comprising:

a display portion comprising a touch sensitive surface having at least one activation area operable by the patient;

a memory for storing at least one portion of the medically relevant information wherein each of the at least one portion of the medically relevant information is associated with a particular activation area;

an audio assembly connected to said memory for producing sound, said audio assembly adapted for receiving a selected portion of the medically relevant information from the memory corresponding to the activation area triggered by the patient to convert the medically relevant information into audio form; and

an electronic article surveillance tag secured to a portion of the device for detecting the removal of the device to an off-premise area.

In another aspect of the present invention, there is provided a portable instructional device for communicating medically relevant information to a patient on-premises during the course of treating, diagnosing or preventing disease, symptoms, or conditions thereof, the instructional device comprising:

a display portion comprising a touch sensitive surface having at least one activation area operable by the patient;

a memory for storing at least one portion of the medically relevant information wherein each of the at least one portion of the medically relevant information is associated with a particular activation area;

an audio assembly connected to said memory for producing sound, said audio assembly adapted for receiving a selected portion of the medically relevant information from the memory corresponding to the activation area triggered by the patient to convert the medically relevant information into audio form;

a wireless radio receiver adapted for receiving a paging signal from an external page transmitter; and

an alert mechanism connected to said wireless radio receiver for alerting the patient that a paging signal has been received.

### BRIEF DESCRIPTION OF THE DRAWINGS

The following drawings in which like reference characters indicate like parts are illustrative of embodiments of the invention and are not intended to limit the invention as encompassed by the claims forming part of the application.

FIG. 1 is a perspective view of a medical instructional device for one embodiment of the present invention;

FIG. 2 is a perspective view of the medical instructional device in an open viewing position in accordance with the present invention;

FIG. 3 is an electrical circuit schematic of an internal central circuitry designed to implement the audio communication feature of the medical instructional device for one embodiment of the present invention;

FIG. 4 is a perspective view from the bottom of the medical instructional device in accordance with the present invention;

FIG. 5 is an assembly view of the medical instructional device detailing the arrangement of the parts in accordance with the present invention;

FIG. 6 is a top plan view of a medical instructional device in the closed position for another embodiment of the present invention;

FIG. 7 is a simplified block diagram of incorporating a medical instructional device into an on-premises paging system for another embodiment of the present invention; and

FIG. 8 is a simplified functional diagram of one of the plurality of medical instructional devices in the system of FIG. 6 in accordance with the present invention.

### DETAILED DESCRIPTION OF THE INVENTION

The present invention is directed to a portable instructional device designed as a hand-holdable information conveying tool for patients prescribed to receive medications, medical devices and treatments, and the like. The instructional device of the present invention is provided to assist physicians and pharmacists in educating their patients on details of a particular medication, treatment option, and medication condition, for example. The instructional device of the present invention provides visual information and audio communication upon activation by the patient. The visual and audio information may be coordinated to provide comprehensive information regarding medication, treatment, medical devices, and the like, to the patient. The information may be provided in at least one language selected by the patient. The instructional device of the present invention may be used advantageously by patients with limited sight, limited literacy, and limited language fluency to learn and educate themselves in a more effective and meaningful manner. In addition, the small size and compact profile of the device makes it especially convenient for carry and portable operation.

In general, the instructional device is preferably dispensed to the patient for use on-premises in anticipation of receiving medically relevant information such as information concerning a medication, medical treatment, medical device and/or medical consultation. The patient may use the instructional device while waiting at the physician's office or at the pharmacy. The instructional device includes an electronic tag or sensor used in electronic article surveillance systems to ensure that the device remains in an on-premise area. Optionally, the instructional device may further include a paging mechanism responsive to a master transmitter or central controller station for paging a patient on-premises.

The instructional device of the present invention has been designed to enhance the patient's retention of information conveyed interactively via visual and audio means. In the context of medication and treatment compliance, the instructional device of the present invention enhances the ease and regularity of administration and improves the patient's ability to remember important information. In addition, the small size and compact profile of the instructional device facilitate carry and portability. The instructional device of the present invention is inexpensive to construct and implement, and is especially suitable for patients including the elderly and those with limited vision or those having difficulty reading fine print material, or those with limited language proficiency or literacy skills.

The instructional device of the present invention will be described generally in context of on-premise usage for educating and instructing patients about medical products such as medications, medical devices, and the like, and for enhancing patient compliance and facilitating proper usage, dosing, and treatment of such products. As used herein, the term “medical product and/or treatment” shall refer to any product, device, medication, and treatment procedure or regimen useful to treat, diagnose, or prevent a medical disease, symptom, or condition thereof. It will, however, be understood that the instructional device of the present invention can be applied to any article of manufacture or service where the consumer will benefit from the furnishing of medically relevant information as defined herein and details about the article of manufacture or service through a coordinated visual and audio means in an “on-premises” setting.

It has been observed through numerous physician/patient interactions that patients are aware of medically relevant information concerning a product (e.g., a medication or a medical device) or medical treatment procedure or regimen, tend to be twice as compliant as compared to those that lacked access to the same information. To enhance compliance, such medically relevant information must be easy for a layperson to understand, and include at least one or more of the following informational targets: 1) details about the product including manner of action, 2) details about proper practice including mode of administration, dosing or usage intervals, and the like, 3) details about duration of usage including length of the prescribed treatment, 4) details about adverse side effects or contraindications, and 5) details about the consequences of terminating use of the product before completion of the prescribed treatment.

As used herein, the term “medically relevant information” is intended to include any information which the patient would find useful in the treatment, diagnosis or prevention of a medical issue such as a disease, symptom or condition thereof in which the patient's health, future or present, is under consideration. Medically relevant information is information that pertains directly or indirectly to, for example, medications (i.e., prescribed and over-the-counter), medical treatments, medical devices, medical tests, preventive medical treatments, diet, healthcare formulations including vitamins, minerals, nutraceuticals, non-traditional therapies (e.g., acupuncture) and the like, drug information, details about affected body parts or organ systems, administration instructions and dosing, symptoms and conditions to be treated, warnings, counter-indications, ingredients, manner of action, proper practice, duration of usage, adverse side effects or risks, consequences of improper usage including premature termination of usage, and the like.

The present invention is not intended to be a substitute for or replace actual medical consultation with medical personnel including doctors, medical supply specialists, and pharmacists. Instead the present invention is designed to be used as a tool or aid to assist the medical profession and particularly medical personnel in providing sufficient disclosure of the medically relevant information to the patient. After using the device of the present invention, the patient may discuss any questions or issues that the patient may have with the attending medical personnel as needed. In addition, the instructional device of the present invention is further designed to carry out such functions in a discreet private manner, thereby ensuring the patient's medical privacy.

The instructional device of the present invention has been designed to coordinate information expressed through visual and audio means to enhance comprehension and retention of information conveyed about a medical product and/or treatment. As used herein, the term “coordination of visual and audio information” or related term means that the visual and audio means of the present device both provide information related to the medical product and/or treatment. The information may be duplicative, complementary, sequential, or the like, so long as the coordination of both forms of information provides a benefit beyond that obtained from the use of audio or visual information alone. By way of example, the audio means may describe in words the dosage regimen required for a particular medication while the visual means provides the same information in printed form. In the context of medication, medical device and treatment compliance, the instructional device of the present invention further encourages the patient to use the information and enhances the patient's capacity to remember the information.

Generally, there is provided a portable instructional device for relaying medically relevant information to patients on-premises in association with a medical treatment, device or medication. In a general embodiment, the instructional device comprises a display portion comprising a touch sensitive surface having at least one activation area operable by the patient, a memory for storing at least one portion of the medically relevant information wherein each of the at least one portion of the medically relevant information is associated with a particular activation area, an audio assembly connected to the memory for producing sound, the audio assembly adapted for receiving a selected portion of the medically relevant information from the memory corresponding to the activation area triggered by the patient to convert the medically relevant information into audio form, and an electronic article surveillance tag secured to a portion of the device for detecting the removal of the device to an off-premise area.

Optionally, the instructional device of the present invention may include a wireless radio receiver adapted for receiving a paging signal from an external page transmitter, and an alert mechanism connected to the wireless radio receiver for alerting the patient that a paging signal has been received.

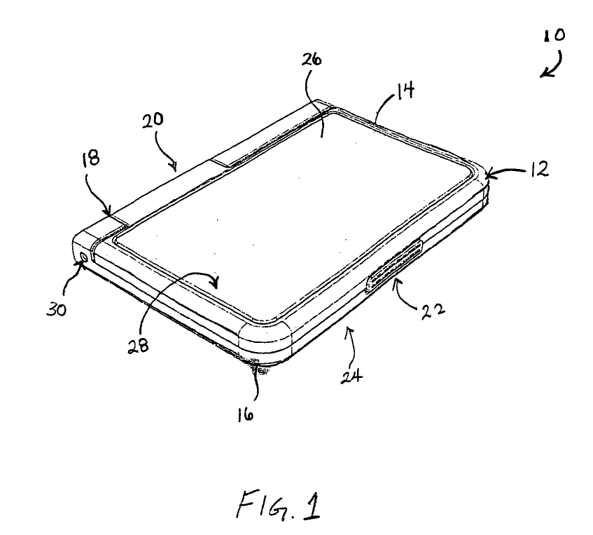

Referring to FIG. 1, a medical instructional device identified generally by reference numeral 10 is shown for one embodiment of the present invention. Typically, a physician, pharmacist, pharmaceutical supply vendor or other medical personnel on the premises will furnish the instructional device 10 to the patient for the purpose of educating or instructing the patient regarding a corresponding prescribed medical device, a prescribed medication, a prescribed medical treatment option, or combinations thereof. The instructional device 10 may be used by the patient on-premises, while waiting for further medical consultation, filling of an order, confirmation of scheduling, preparation of a referral, and the like. In this manner, if the patient has further questions, those questions may be offered to the attending medical personnel.

In one setting, the instructional device 10 is provided to assist elderly patients, patients with limited or no vision, patients with limited literacy skills, and/or patients with limited language fluency. The instructional device 10 is further designed to convey information in a manner to enhance patient compliance in order to prevent adverse effects such as injury or death, and minimize ineffective treatments through improper or incomplete use. For those not fluent in a specific language, the information may be conveyed in a language the user may better understand.

The instructional device 10 is designed to promote effective interaction with the patient in association with the medical product and/or treatment. The instructional device 10 may be constructed of any durable material including plastic, and molded into any size, shape, or color for easy identification and association with a specific product, medication or treatment option.

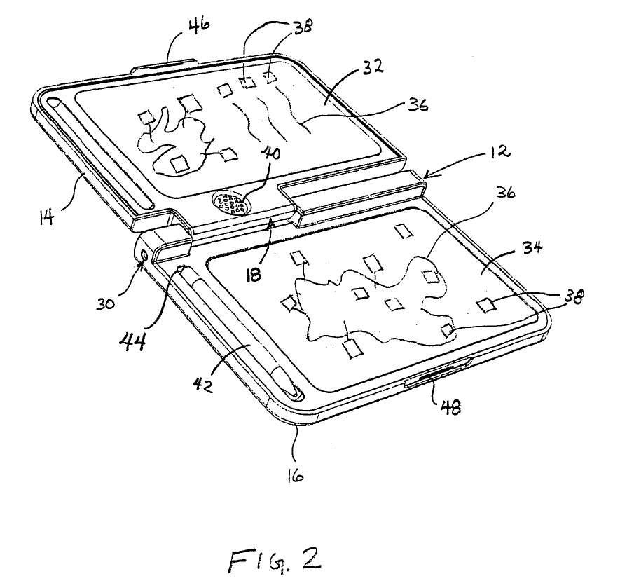

The instructional device 10 comprises a housing 12 composed of a first panel portion 14, a second panel portion 16 connected through a hinge 18 located along the rear periphery 20 thereof. The first panel portion 14 includes a display placard receiving area 28 enclosed by a removable cover 26. The instructional device 10 further comprises a latch assembly 22 located on the front periphery 24 thereof. The latch assembly 22 maintains the instructional device 10 in the closed position during storage or non-use. The device 10 further includes a speaker 40 (see FIG. 2) and an earphone jack 30 located proximate the rear periphery 20 thereof for receiving the plug of an earphone or headset. Optionally, built-in head phones or a head set may be provided for enhancing convenience and accessibility as well as privacy.

Referring to FIG. 2, the latch assembly 22 includes a latch 46 and a corresponding latch engaging recess 48. The instructional device 10 can be moved into an open viewing position by disengaging the latch 46 from the corresponding recess 48, and moving the first panel portion 14 away from the second panel portion 16. The instructional device 10 further comprises two flexible interactive membranes 32 and 34 each overlaying a portion of the corresponding first and second panel portions 14 and 16. The flexible interactive membranes 32 and 34 are composed of a resilient sheet material, and include at least one activation area or key 38, and information 36 in the form of text and graphical depictions (e.g., drug effects, affected body parts, and contraindications) printed on the surface thereof in association with the corresponding activation area 38. The activation area 38 is sensitive to touch or pressure.

The operation of the instructional device 10 is initiated by the patient through the touching or application of pressure on the activation area 38. Each activation area 38 identified by the printed information 36 corresponds to the desired audio information the patient wishes to hear. The pressing of the activation area 38 generates an input from the patient to the instructional device 10. The number and placement of the activation area 38 on the flexible interactive membrane 32 or 34 will typically depend on the arrangement, context and content of the printed information 36.

The usual visual information provided by the printed information 36 on the interactive membranes 32 and 34 and the information contained in the audio communication are coordinated to provide a comprehensive teaching about the medical product and/or treatment to enable the patient to properly employ the same including medically relevant information. The coordination of the visual and audio means may be accomplished by providing instructions by audio with a graphic visual display.

During operation and use, the patient may read the printed information 36 on the interactive membranes 32 and 34, and initiate playback of the corresponding audio communication through triggering of the corresponding activation area 38. The audio communication relating to the printed information 36 is conveyed to the patient, thus providing coordinated comprehensive information to the patient about the medical product and/or treatment including medically relevant information. The audio communication is outputted through the speaker 40 or the head phones port 30.

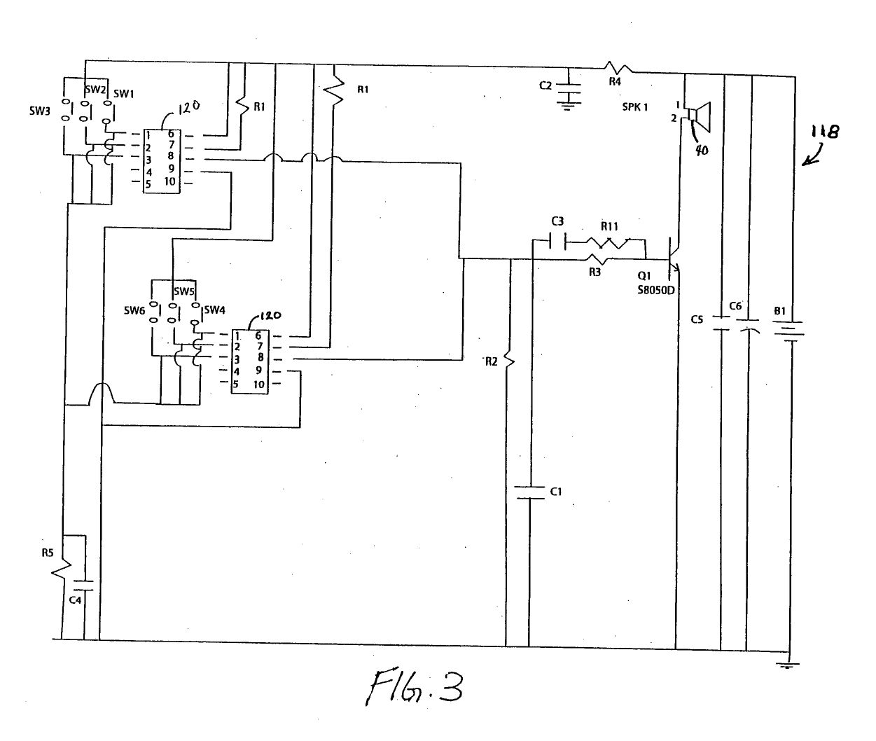

Referring to FIG. 3, the instructional device 10 further includes an internal central circuit 118 programmed for receiving and handling the input from the activation area 38. The internal central circuit 118 responds by identifying the input and effecting the production of a corresponding audio output through the earphone jack 30 or the speaker 40. In this manner, the internal central circuit 118 is configured to identify the input from the patient, determine the corresponding audio output, and effecting the audio output device (i.e., speaker or headset) to generate the audio output in the form of a sound to the patient. As shown in FIG. 2, a stylus 42 located in a holding slot 44 may be provided to assist the patient's selection of the desired activation area 38.

As shown in FIG. 3, the internal central circuit 118 comprises at least one processor 120 in the form of sound chips, preferably digital based sound chips, and corresponding switches SW1-SW6 for initiating input signals, and powered by a voltage or battery source B1. The present internal central circuit 118 is designed to be used with up to six activation areas 38 in connection with the six switches SW1-SW6. It is noted that the present invention is not limited to such number of switches, and the number of switches may vary depending on the number of messages desired to be stored and played back. As used herein, “sound chip” is any integrated circuit designed to produce a sound through various means including digital, analog, and mixed-mode electronics, and is capable of record and/or play sound segments using internal memory such as, for example, erasable programmable read only memory (EPROM) or static random access memory (SRAM), or in combination with external memory sources. A suitable example of a sound chip is the QuikVoice™ line of digital sound chips marketed by Eletech Electronics, Inc., of City of Industry, CA.

The processor 120 may be programmed with pre-set sound segments into its EPROM using a standard EPROM programmer. Once programmed, the processor 120 can be activated by triggering the corresponding switches SW1 to SW6, respectively. The output of the processor 120 is passed through the remaining portions of the internal central circuit 118 comprising resistors R1-R4, and R11, capacitors C1-C6, and amplifier Q1 to furnish a filter to generate an audio sound from the speaker 40.

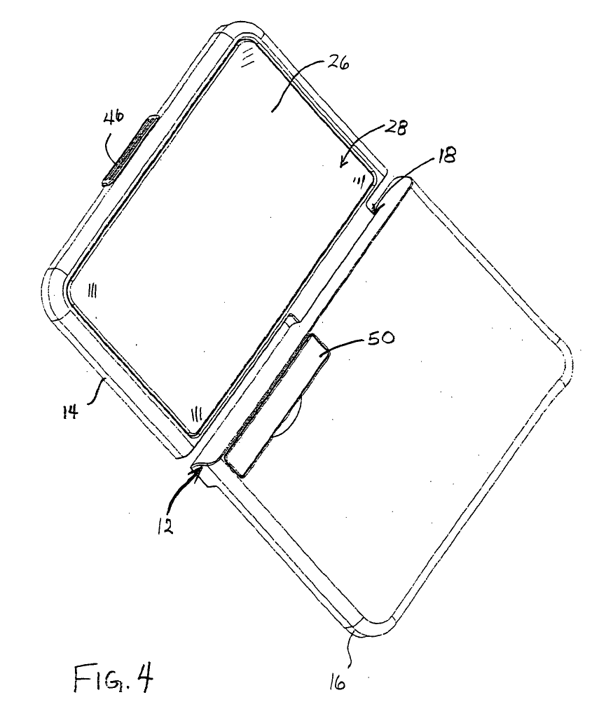

Referring to FIG. 4, the exterior portion of the instructional device 10 is shown in the open viewable position. The internal central circuit 118 and the various components are powered by a battery source B1 of FIG. 3. The second panel portion 16 includes a battery access door 50 to allow the user to replace the battery source B1 as required. The first panel portion 14 of the device 10 includes the display placard receiving area 28 suitable for reversibly receiving and accommodating a display placard (not shown). The display placard receiving area 28 is secured by the removable cover 26 to retain the display placard thereon. The removable cover 26 is generally see-through or transparent through which the patient can view the retained display placard. Additionally, the cover 26 may be sufficiently rigid to allow the patient to use this portion of the device 10 as a writing surface upon which the user can make notes or fill out forms, for example.

The display placard is typically printed with information identifying the subject matter stored in the device 10. The printed information may communicate to the patient any information including, but not limited to drug information, administration instructions and dosing, symptoms and conditions to be treated, warnings, active ingredients, and the like. In the alternative, the patient may use the device 10 as a writing surface upon which to write notes or fill out forms, for example.

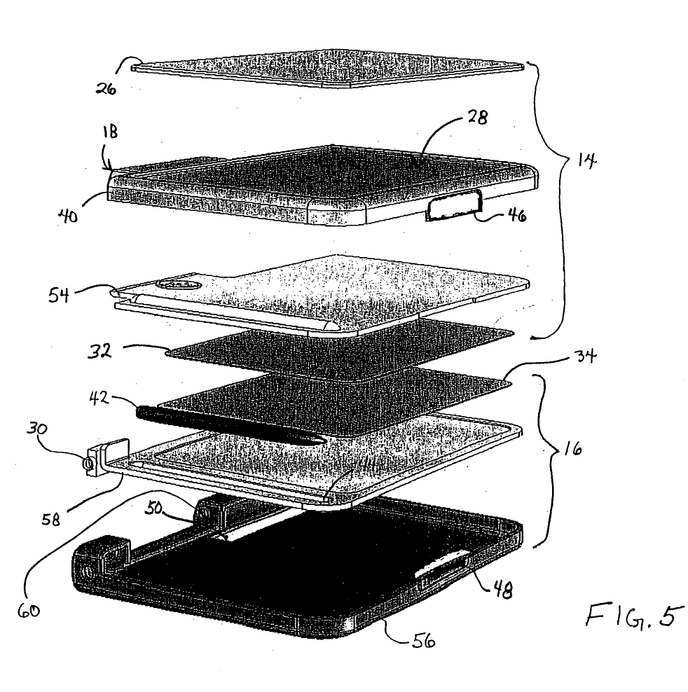

Referring to FIG. 5, the first panel portion 14 comprises a first panel shell 40 having the display placard receiving area 28 enclosed by the removable cover 26, a first support plate 54 supporting the speaker 40 and enclosing the shell 40, and the flexible interactive membrane 32 overlaying a surface 60 of the plate 54. The second panel portion 16 comprises a second panel shell 56 for accommodating the internal central circuit 118, a second support plate 58 holding the earphone jack 30 and enclosing the shell 56, and the flexible interactive membrane 34 overlaying the surface 62 of the plate 58. The first panel portion 14 and the second panel portion 16 are attached to one another through the coupling of the hinge 18 of the first panel shell 40 and a corresponding hinge receiving portion 60 of the second panel shell 56.

The instructional device 10 includes a number of pressure transducers or sensors (not shown) each corresponding to an activation area 38 of the flexible interactive membrane 32 or 34. The pressure sensors (not shown) in operative association with the activation areas 38, are located between the flexible interactive membranes 32 and 34 and the corresponding support plates 54 and 58.

As previously described, the flexible interactive membranes 32 and 34 are composed of a resilient sheet material that bends in response to mechanical pressure applied thereon. In this manner, when pressure is applied to the activation areas 38, the corresponding portions of the membrane 32 or 34 deforms and causes the corresponding pressure sensor to become activated. The pressure sensors (not shown) are electrically connected to the internal central circuit 118, and are adapted to generate a signal to the internal central circuit 118 when the patient presses the corresponding activation area 38. Such pressure transducers or sensors are known in the art and typically function to generate an electrical output in response to the application of a mechanical force or pressure.

Alternatively, it is noted that the instructional device may be designed having the flexible interactive membrane, pressure sensors and support plate replaced with an touch interactive video display unit such as, for example, a liquid crystal display in combination with a touch screen, a touch panel or a touch screen panel mechanism. The video display unit may be used to display a series of video screens, which the user can use and interact through touch to request audio information. Such touch screens are typically pressure-sensitive (resistive), electrically-sensitive (capacitance), acoustically-sensitive (SAW—surface acoustic wave) or photo-sensitive (infra-red). The effect of such overlays allows the display to be used as an input device.

Referring generally to FIGS. 1-5, the general operation of the instructional device 10 will be described. The instructional device 10 is initiated by the triggering of the activation areas 38 of flexible interactive membrane 32 and 34, and the corresponding pressure sensors (not shown) represented generally as an input device. Upon activation by the patient, the input device generates an input signal corresponding to the pressed activation area 38. The input signal is transmitted to the processor 120 in the form of a digital sound chip of the internal central circuit 118 in FIG. 3. The processor 120 identifies the input signal and correlates with a memory location storing at least one audio message embodied in an electronic form. Upon retrieving the corresponding audio message, the processor 120 outputs the electronic message to an audio speaker 40. The audio speaker 40 processes electronic message into an output signal where either the headset connected to the earphone jack 30, or the speaker 40 generates from the output signal the audio message as a sound for the patient to hear.

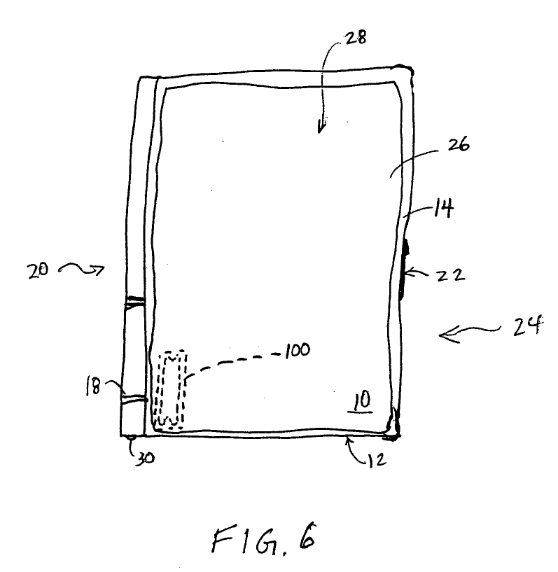

Referring to FIG. 6, the instructional device 10 further includes an electronic article surveillance or EAS tag 100 which enables detection of the removal of the instructional device 10 from an on-premise area by the patient. The EAS tag 100 is generally mounted to an interior portion of the instruction device 10, preferably within the housing 12. The EAS tag 100 ensures that the instructional device 10 remains on the premises as intended for use by patients. The EAS tag 100 includes a side portion which is secured to an inner surface of the housing 12 through an adhesive or the like. The EAS tag 100 may be selected from radio frequency EAS tags, electromagnetic EAS tags, and acousto-magnetic EAS tags, and more preferably acousto-magnetic EAS tags. A suitable acousto-magnetic EAS tag is marketed by Sensormatic Electronics Corporation of Boca Raton, Fla., under the trademark ULTRA-STRIP III. Such EAS tags can be sensed or detected by commercially available detectors at the on-premise area exits to ensure that the instructional device 10 remains on the premises as intended.

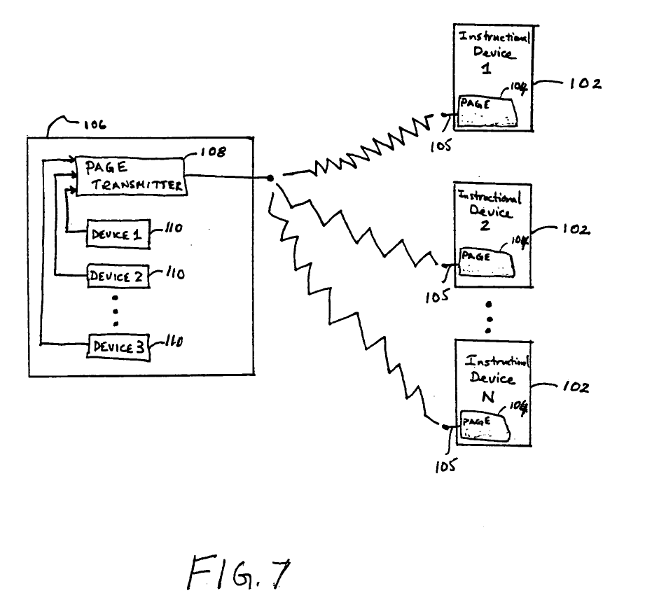

Referring to FIG. 7, an instructional device 102 is shown for a second embodiment of the present invention. The instructional device 102 comprises all the features shown and described in connection with the instructional device 10. The instructional device 102 further includes a page mechanism 104 with a page receiver 105. The page mechanism 104 is constructed to operate with a centralized control station 106. The centralized control station 106 includes a plurality of buttons 110 which may be pressed individually to send a page via a page transmitter 108 to one of an associated plurality of instructional devices 102.

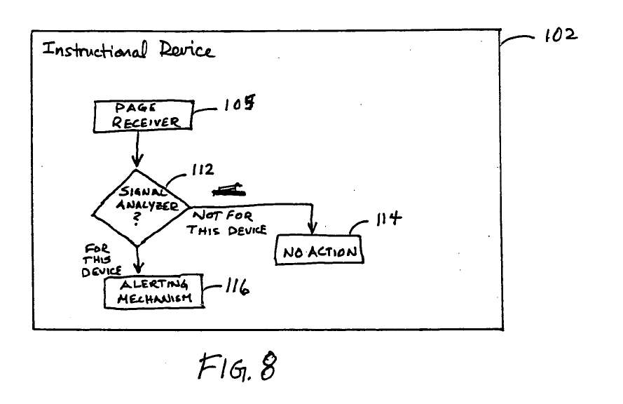

Referring to FIG. 8, the instructional device 102 of FIG. 7 includes the page receiver 15 and a signal analyzer 112 which determines whether a received signal is intended for that device. If the received signal is not intended for that device, the device takes no action as shown in 114. If the received signal is intended for that device, the device activates an alerting mechanism 116 which visually, audibly or tactily alerts the patient that a page has been received. The alert mechanism 116 may alert the patient through a blinking light, a vibration generator, a beep or an alphanumeric display.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A portable instructional device for communicating medically relevant information to a patient on-premises in the course of treating, diagnosing or preventing disease, symptoms, or conditions thereof, said instructional device comprising:
a display portion comprising a touch sensitive surface having at least one activation area operable by the patient;
a memory for storing at least one portion of the medically relevant information wherein each of the at least one portion of the medically relevant information is associated with a particular activation area;
an audio assembly connected to said memory for producing sound, said audio assembly adapted for receiving a selected portion of the medically relevant information from the memory corresponding to the activation area triggered by the patient to convert the medically relevant information into audio form; and
an electronic article surveillance tag secured to a portion of the device for detecting the removal of the device to an off-premise area.

2. The instructional device of claim 1 further comprising:
a wireless radio receiver adapted for receiving a paging signal from an external page transmitter; and
an alert mechanism connected to said wireless radio receiver for alerting the patient that a paging signal has been received.

3. The instructional device of claim 1 further comprising printed information on the display portion corresponding to the at least one activation area, and coordinated with the at least one portion of the medically relevant information.

4. The instructional device of claim 1 wherein the medically relevant information is selected from the group consisting of manner of action, proper practice, duration of usage, adverse side effects or risks, consequences of prematurely ceasing usage, and combinations thereof.

5. The instructional device of claim 1 wherein the audio assembly further comprises a port for receiving a headset device.

6. The instructional device of claim 1 further comprising at least one panel supporting the display portion.

7. The instructional device of claim 6 wherein the at least one panel comprises two panels hingedly attached one another along an edge portion wherein the panels are pivotably movable from a juxtaposed closed position to an open book-like position.

8. The instructional device of claim 1 further comprising a hand-holdable stylus for pressing the at least one activation area.

9. The instructional device of claim 1 further comprising a display placard receiving area enclosed by a see-through cover located thereon for receiving and reversibly retaining a display placard.

10. The instructional device of claim 1 further comprising a writing surface for supporting a writing instrument and substrate on which the patient can write.

11. A portable instructional device for communicating medically relevant information to a patient on-premises during the course of treating, diagnosing or preventing disease, symptoms, or conditions thereof, said instructional device comprising:
a display portion comprising a touch sensitive surface having at least one activation area operable by the patient;
a memory for storing at least one portion of the medically relevant information wherein each of the at least one portion of the medically relevant information is associated with a particular activation area;
an audio assembly connected to said memory for producing sound, said audio assembly adapted for receiving a selected portion of the medically relevant information from the memory corresponding to the activation area triggered by the patient to convert the medically relevant information into audio form;
a wireless radio receiver adapted for receiving a paging signal from an external page transmitter; and
an alert mechanism connected to said wireless radio receiver for alerting the patient that a paging signal has been received.

12. A method for furnishing medically relevant information to a patient on-premises during the course of treating, diagnosing or preventing disease, symptoms, or conditions thereof, said method comprising dispensing the portable instructional device of claim 1 to the patient.

13. A method for furnishing medically relevant information to a patient on-premises during the course of treating, diagnosing or preventing disease, symptoms, or conditions thereof, said method comprising dispensing the portable instructional device of claim 11 to the patient.
